(19) United States
(12) Patent Application Publication
(10) Pub. No.: US 2019/0246970 A1
(43) Pub. Date: Aug. 15, 2019

(54) SHEATH PRODUCT, MANUFACTURING PROCESS AND EQUIPMENT THEREOF, AND DATA COLLECTION SYSTEM HAVING SHEATH PRODUCT

(71) Applicants: Rulin Chen, Guangzhou, Guangdong (CN); Victor W J Chan, Guangzhou, Guangdong (CN)

(72) Inventors: Rulin Chen, Guangzhou, Guangdong (CN); Victor W J Chan, Guangzhou, Guangdong (CN)

(21) Appl. No.: 15/894,969

(22) Filed: Feb. 13, 2018

(51) Int. Cl. A61B 5/00; A61B 5/11; A61B 5/0205

(52) U.S. Cl. CPC A61B 5/4393; A61B 5/11; A61B 5/02438; A61B 5/002; A61B 5/02055

(57) ABSTRACT

Provided are a sheath product with sensing function, a manufacturing process of the sheath product, a manufacturing equipment of the sheath product, and a data collecting system having the sheath product. The sheath product comprises a sheath body, the sheath body is provided with a pre-curling region, the pre-curling region is rolled up outwards to form a cuff in which a sensing chip is embedded.

### FIELD OF THE INVENTION

This invention relates to the field of healthcare, more particularly to a sheath product with sensing function, a manufacturing process of the sheath product, a manufacturing equipment of the sheath product, and a data collecting system having the sheath product.

### BACKGROUND OF THE INVENTION

Condoms, also known as prophylactics, are mainly used for birth control and prevention of sexually transmitted diseases. Since the time it takes to reach orgasm for a male may be different from that for a female sexual partner, the mentioned function of conventional condoms cannot simultaneously satisfy both partners' sexual needs.

Conventional condoms are unable to collect and analyze data of user's physical characteristic or user's body movement in real time, which leads to user's inability of acknowledging their body condition during sexual intercourse therefore controlling the pace of their sex life. Additionally, lacking a system combining a condom with an intelligent terminal under current technology, users are unable to directly and accurately obtain the data revealing their body condition during sexual intercourse and adjust their body in the light of the data regarding their physical characteristic and body movement to control their sex life. This can easily cause sex apathy in female or impotence and premature ejaculation etc. in male, which ultimately leads to unsatisfactory sex.

### SUMMARY OF THE INVENTION

The present invention aims at providing a sheath product with sensing function, a manufacturing process of the sheath product, a manufacturing equipment of the sheath product, and a data collection system having the sheath product to overcome the shortcomings of existing condoms in the above-mentioned prior arts.

In order to solve the above-mentioned technical issues, the first aspect of the present invention provides a manufacturing process of embedding a sensing chip in a sheath product comprising: in a production line of manufacturing a sheath product, dipping a mold designed according to a shape of the sheath product in a solution of vulcanized rubber; pulling out the mold from the solution of vulcanized rubber and drying it to form at least one layer of rubber film on the mold; mounting a sensing chip onto a pre-curling region of the rubber film by a chip mounter before rolling up the pre-curling region of the rubber film formed on the mold; and rolling up outwards the pre-curling region by a beading machine to form a cuff so as to complete the manufacturing of the sheath product with sensing function.

Preferably, after the chip mounter mounting the sensing chip onto the pre-curling region of the rubber film but prior to rolling up the pre-curling region of the rubber film on the mold, there is another mold dipping process in which an exterior surface of the rubber film mounted with the sensing chip is formed with at least one additional layer of rubber film.

Preferably, the sensing chip is a flexible sensing chip and is rolled up outwards with the pre-curling region.

Preferably, the sensing chip is attached on the pre-curling region of the rubber film horizontally, with a longitudinal direction of the sensing chip being consistent with a circumferential direction of the pre-curling region; or the sensing chip is attached on the pre-curling region of the rubber film vertically, with the longitudinal direction of the sensing chip being consistent with a longitudinal direction of a sheath body; or the sensing chip is attached on the pre-curling region of the rubber film slantwise, with the longitudinal direction of the sensing chip being tilted against the longitudinal direction of the sheath body; or the sensing chip is ring-shaped, which surrounds and attaches on an exterior circumferential surface of the pre-curling region of the rubber film.

The second aspect of the present invention provides a manufacturing equipment for embedding a sensing chip into a sheath product, comprising: a sheath-product production line; at a tail of the sheath-product production line, a chip mounter configured for mounting a sensing chip onto a pre-curling region of a rubber film; and at a tail of the chip mounter, a beading machine configured for rolling up the pre-curling region outwards to form a cuff.

Preferably, the sensing chip is a flexible sensing chip and is rolled up outwards with the pre-curling region.

The third aspect of the present invention provides a sheath product with sensing function, wherein the sheath product comprises a sheath body, the sheath body is provided with a pre-curling region, the pre-curling region is rolled up outwards to form a cuff in which a sensing chip is embedded.

Preferably, the sheath body is made of rubber, latex or silicone.

Preferably, the sensing chip is a flexible sensing chip and is rolled up outwards with the pre-curling region, the sensing chip is an electrical element which senses user's physical characteristic data and/or user's movement data in real time and transmits such physical characteristic data and/or movement data in real time.

Preferably, the sensing chip comprises a power supply module, a sensing module and a data transmission module; wherein the power supply module provides power to the sensing module and the data transmission module by self-contained power supply or wireless power supply; the sensing module senses user's physical characteristic data and/or user's movement data; the user's physical characteristic data comprises body temperature, pulse rate, respiration, and blood pressure, while the user's movement data comprises velocity, acceleration, and pressure; the data transmission module transmits data received from the sensing module to an intelligent terminal by wireless communication channel.

The fourth aspect of the present invention provides a data collection system, wherein the data collection system comprises an intelligent terminal and the sheath product with sensing function mentioned above; the sheath product with sensing function senses user's physical characteristic data and/or user's movement data and transmits such physical characteristic data and/or movement data to the intelligent terminal in real time; the intelligent terminal receives user's physical characteristic data and/or user's movement data delivered by the sheath product and processes as well as displays such physical characteristic data/or movement data.

Preferably, the intelligent terminal supplies wireless power to or charges the power supply module of the sensing chip of the sheath product.

In conclusion, the beneficial effects of the embodiments of the present invention compared to prior arts are as follows.

Firstly, the present invention adopts a process and an equipment for embedding a sensing chip, which are working with the sheath-product production line to realize automatic continuous production of sheath products. This breaks through the limitation of implementing intelligent sheath products and solves the technical issue as to embedding sensing chips into sheath products.

Secondly, the present invention adopts a protective and concealed technique to embed sensing chips into sheath products, which is working with the curling technique and the vulcanization technique to avoid any adverse influence on the appearance of the sheath products after rolling up the sensing chip into the pre-curling region. This technique is reliable as it avoids the sheath product falling off or getting damaged due to friction or impact caused during any movements occurred in usage.

Thirdly, the present invention makes it possible to collect user's physical characteristic data and/or user's movement data via the sensing chip mounted in the cuff of the sheath body. Wherein the user's physical characteristic data includes but is not limited to body temperature, pulse rate, respiration and blood pressure and the user's movement data includes but is not limited to velocity, acceleration and pressure. Such collected data are then transmitted to the intelligent terminal via wireless communication channel. Wherein the wireless communication channel comprises Bluetooth, Wi-Fi (Wireless Fidelity) and various wireless sensing techniques such as RFID (Radio Frequency Identification) etc. Users are able to obtain their physical characteristic data and/or movement data through external smart devices directly, accurately and in real time. These data enable the users to manage and adjust their body condition to control the pace of their sex life in real time, which consequently improves their experience.

### BRIEF DESCRIPTION OF THE DRAWINGS

In order to give a clearer illustration of the embodiments of the present invention, a brief description of the drawings is set out as follows.

FIG. 1 is a schematic diagram showing a sheath product with sensing function according to a preferred embodiment of the present invention.

FIG. 2 is a block diagram of a sensing chip of a sheath product with sensing function according to an embodiment of the present invention.

FIG. 3 is a schematic diagram showing a system collecting and analyzing user's physical characteristic data and/or user's movement data according to an embodiment of the present invention.

FIG. 4 is a schematic diagram showing data interaction between a sheath product with sensing function of the present invention and an intelligent terminal.

FIG. 5 is a schematic diagram showing a power supply module according to an embodiment of the present invention.

FIG. 6 is a schematic diagram showing a sensing module according to an embodiment of the present invention.

FIG. 7 is a flow chart showing a manufacturing process of making a sheath product with sensing function according to an embodiment of the present invention.

FIG. 8 is a schematic diagram showing a sheath product with sensing function according to another preferred embodiment of the present invention.

FIG. 9 is a schematic diagram showing a first manner in which a sensing chip is mounted to a pre-curling region according to an embodiment of the present invention.

FIG. 10 is a schematic diagram showing a second manner in which a sensing chip is mounted to a pre-curling region according to another embodiment of the present invention.

FIG. 11 is a schematic diagram showing a third manner in which a sensing chip is mounted to a pre-curling region according to another embodiment of the present invention.

FIG. 12 is a schematic diagram showing a fourth manner in which a sensing chip is mounted to a pre-curling region according to another embodiment of the present invention.

### DETAILED DESCRIPTION OF ILLUSTRATED EMBODIMENTS

Embodiments of the present invention are further explained clearly as follows in conjunction with figures. Apparently, the following embodiments are mere a part of rather than all of embodiments of the present invention. Based on the embodiments of the present invention, all or any other embodiments gained without innovative efforts made by the skilled in the art are regarded as falling within the scope of the protection to the present invention.

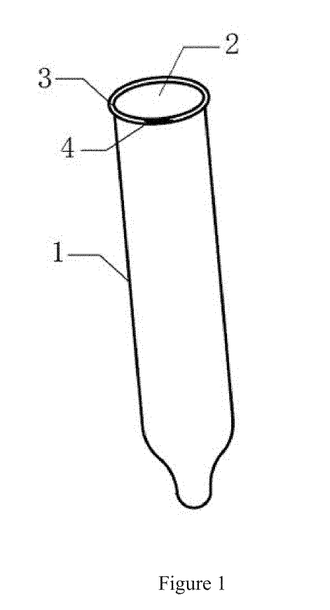

As shown in FIG. 1, which is a schematic diagram illustrating a preferable embodiment of a sheath product 100 with sensing function provided by the present invention.

In the present invention, the sheath product 100 with sensing function comprises a sheath body 1. The sheath body 1 is provided with a pre-curling region 2 (i.e., an open end). The pre-curling region 2 is rolled up outwards to form a cuff 3. A sensing chip 4 is embedded in the cuff 3.

Preferably, the sensing chip 4 is a flexible sensing chip, which is capable of being rolled up outwards with the pre-curling region 2.

FIG. 9 shows a first manner in which the sensing chip 4 is mounted to the pre-curling region 2. Prior to rolling up the sensing chip 4 with the pre-curling region 2, the sensing chip 4 is preferably to be ring-shaped, which surrounds and mounted to the pre-curling region 2. The sensing chip 4 may be mounted to an exterior or inner circumferential surface of the pre-curling region 2, or the sensing chip 4 may be inserted in an interior of the pre-curling region 2. Then the sensing chip 4 is rolled up outwards with the pre-curling region 2 to form the cuff 3. Apparently, the concurrency in rolling up both the pre-curling region 2 and the sensing chip 4 gives rise to complete avoidance of increasing thickness of any part of the sheath body 1, local crowning and unfavorable sensation.

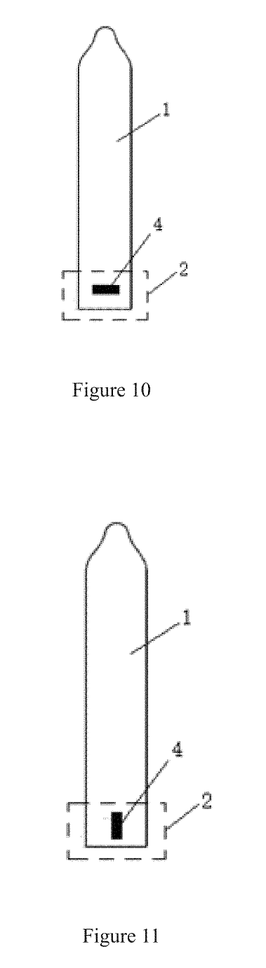

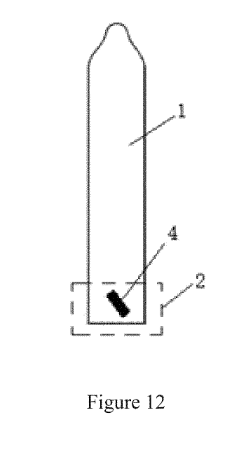

FIGS. 10-12 show other manners in which the sensing chip 4 is mounted to the pre-curling region 2. For the convenience of the embedment of the sensing chip 4 in the cuff 3, the sensing chip 4 is mounted to the pre-curling region 2 horizontally prior to rolling up the sensing chip 4 with the pre-curling region 2, i.e., the sensing chip 4 is mounted to the pre-curling region 2 with its longitudinal direction consistent with a circumferential direction of the pre-curling region 2 (with reference to FIG. 10). Also, the sensing chip 4 could be mounted to the pre-curling region 2 vertically, i.e., the sensing chip 4 is mounted to the pre-curling region 2 with its longitudinal direction consistent with a longitudinal direction of the sheath body 1 (with reference to FIG. 11). Alternatively, the sensing chip 4 is mounted to the pre-curling region 2 slantwise, i.e., the sensing chip 4 is mounted to the pre-curling region 2 with its longitudinal direction tilted against the longitudinal direction of the sheath body 1 (with reference to FIG. 12). What should be stressed here is that all these above-mentioned designs will lead to increase of thickness due to rolling up the sensing chip 4, which will consequently cause local crowning on the cuff 3.

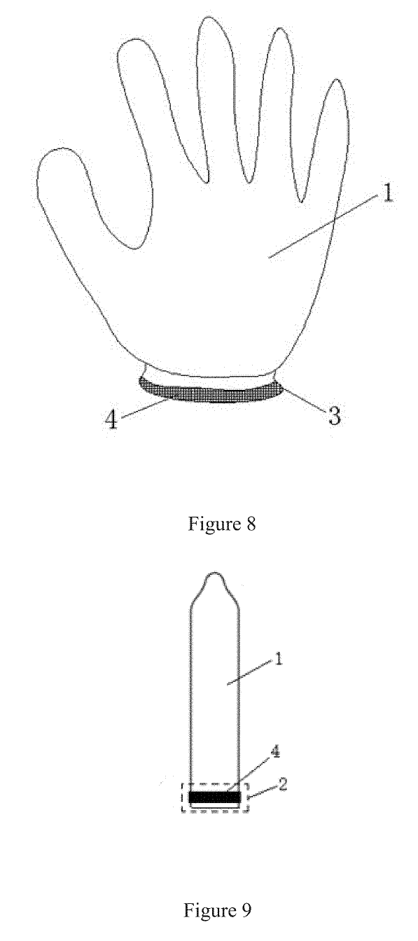

Preferably, the sheath product 100 is condom, glove, headgear or the like made of rubber, latex or silicone. As shown in FIG. 8, the sheath product 100 with sensing function in this embodiment is a glove.

Preferably, the sensing chip 4 is an electrical element that is able to sense user's physical characteristic data and/or user's movement data in real time and transmit such physical characteristic data and/or movement data in real time.

Specifically speaking, the user's physical characteristic data includes but is not limited to one of or a combination of body temperature, pulse rate, respiration and blood pressure. The user's movement data includes but is not limited to one of or a combination of velocity, acceleration and pressure.

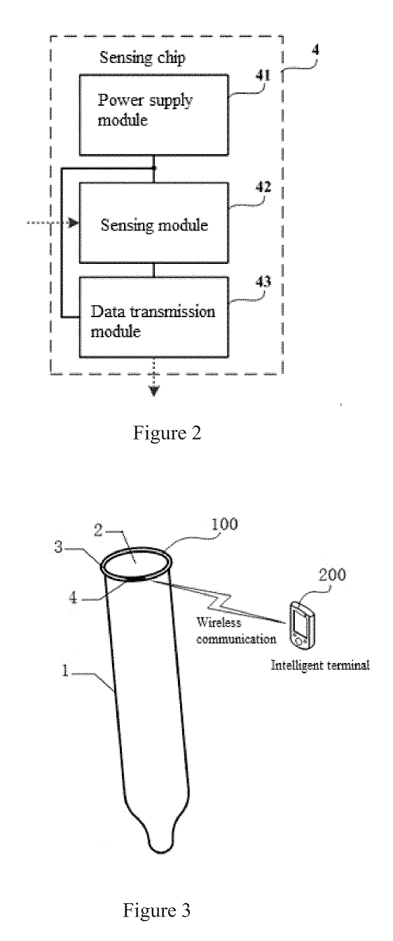

FIG. 2 is a block diagram of the sensing chip 4 of the sheath product with sensing function provided by the present invention.

Specifically speaking, the sensing chip 4 comprises a power supply module 41, a sensing module 42 and a data transmission module 43 that are connected to a flexible circuit board.

The power supply module 41 provides power to the sensing module 42 and the data transmission module 43 by self-contained power supply or wireless power supply. Specifically in FIG. 5, the power supply module 41 is a flexible cell. The flexible cell is a strip. The flexible cell comprises a plurality of electrically-connected flexible cell units. Each of the flexible cell unit comprises, from its top to its bottom, a light-incidence electrode layer 411, a photoelectric conversion layer 412, a non-light-incidence electrode layer 413 and a conductive substrate 414. Each of the flexible cell unit comprises a frontal cut-into surface 415 with its tip 418 facing the conductive substrate 414 and a back cut-into surface 416 with its tip 419 facing the light-incidence electrode layer 411. The frontal cut-into surface 415 of each flexible cell unit faces the back cut-into surface 416 of a neighboring flexible cell unit. The flexible cell further comprises a conductive material 417 wherein the conductive material 417 conductively connects the light-incidence electrode layer 411 of each flexible cell unit with the conductive substrate 414 of each neighboring flexible cell unit.

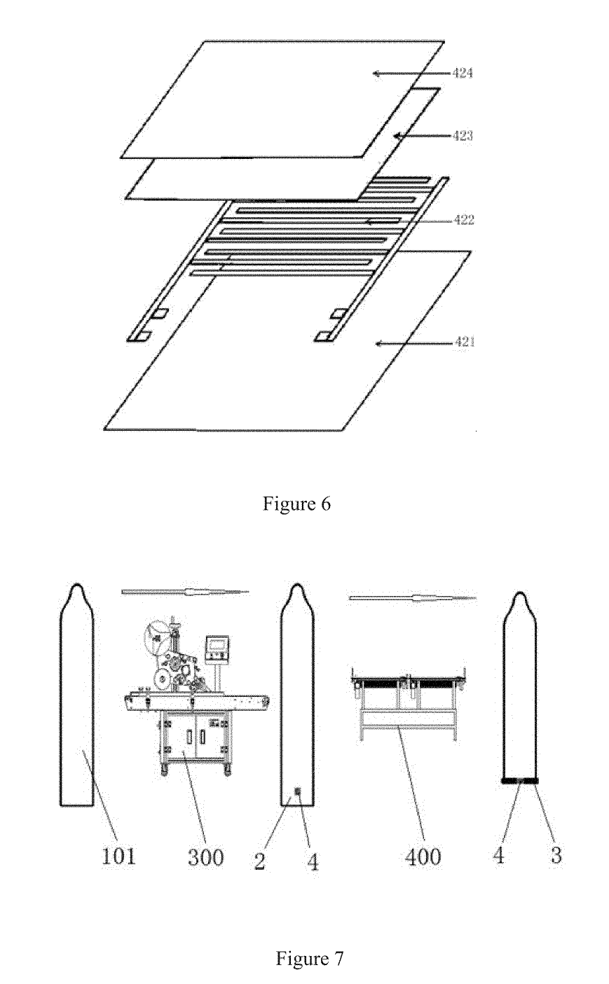

The sensing module 42 comprises a sensor sensing user's physical characteristic data and/or user's movement data. Wherein the user's characteristic data includes but is not limited to body temperature, pulse rate, respiration, and blood pressure. The user's movement data includes but is not limited to velocity, acceleration, and pressure. Specifically speaking, as shown in FIG. 6, the sensing module 42 is a strip and wraps around an inner circumferential surface of the cuff 3 along its longitudinal direction such that the sensing module 42 is close to the human body. The sensing module 42 comprises a metal layer 422, a sensor layer 423 and a passivation layer 424 that are placed in order on an insulative substrate 421. The sensor layer 423 is electrically connected to the metal layer 422 via a terminal. The metal layer 422 is electrically connected to the power supply module 41 and the data transmission module 43 respectively via a circuit on the flexible circuit board. The sensor layer 423 may adopt a thermal resistor when it is used for sensing body temperature where a piezoelectric semiconductor resistor may be adopted when the sensor layer 423 is configured for sensing pulse rate, blood pressure, velocity and the alike data.

The data transmission module 43 is configured to transmit data to an intelligent terminal by wireless communication channel such as Bluetooth, Wi-Fi (Wireless Fidelity) and various wireless sensing techniques such as RFID (Radio Frequency Identification), ZigBee and NFC (Near Field Communication).

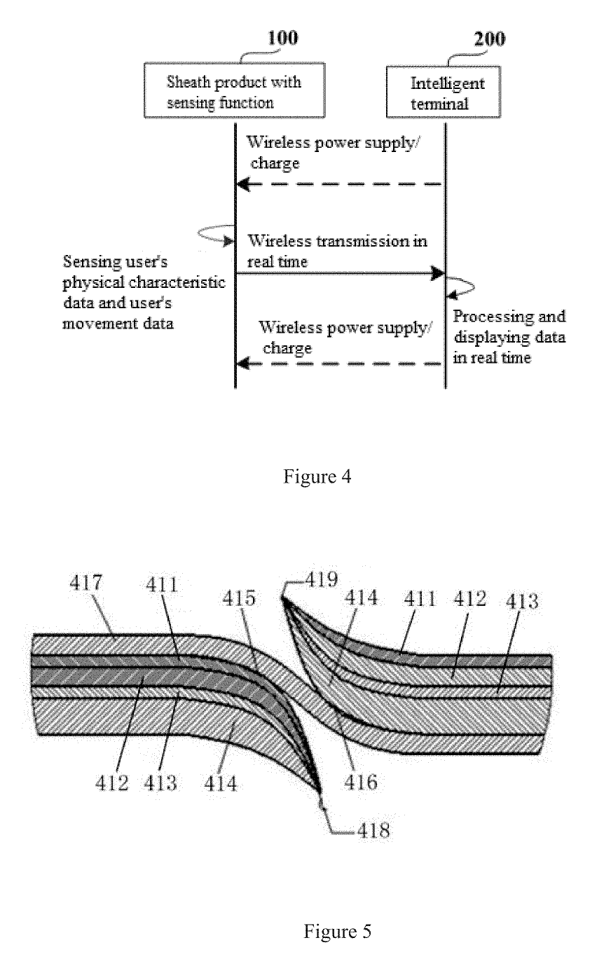

As shown in FIG. 3, corresponding to the above-mentioned sheath product 100 with sensing function, the present invention further provides a system to collect and analyze user's physical characteristic data and/or user's movement data. The system comprises an intelligent terminal 200 and the sheath product 100 with sensing function according to any of the above-mentioned embodiments. Wherein the sheath product 100 with sensing function senses user's physical characteristic data and/or user's movement data and transmits such physical characteristic data and/or movement data to the intelligent terminal 200 in real time. The intelligent terminal 200 receives user's physical characteristic data and/or user's movement data delivered by the above-mentioned sheath product 100 and processes as well as displays such physical characteristic data and/or movement data. In a specific embodiment, as shown in FIG. 4, the sheath product 100 with sensing function collects user's physical characteristic data and/or user's movement data in real time through the sensing chip 4, and then transmits such physical characteristic data and/or movement data to the intelligent terminal 200 via wireless communication channel. Whereas the intelligent terminal 200 processes and displays user's physical characteristic data and/or user's movement data in real time once received such data. Therefore, users are enabled to obtain the data of their body condition directly, accurately and in real time.

Preferably, the intelligent terminal 200 supplies wireless power to or charges the power supply module 41 of the sensing chip 4 of the sheath product 100. Therefore, the intelligent terminal 200 is able to provide wireless power to the power supply module 41 of the sensing chip 4 of the sheath product 100 before or during the usage of the sheath product 100.

In specific embodiment, the intelligent terminal 200 comprises but is not limited to smart phones, tablets, portable computers and handheld smart devices etc.

Corresponding to the above-mentioned sheath product 100 with sensing function, the present invention further provides a manufacturing process in relation to embedding a sensing chip in such sheath product, which is illustrated in FIG. 7.

During the production line of manufacturing a sheath product, firstly dipping a mold designed according to the shape of the sheath product in a solution of vulcanized rubber. Then pull out the dipped mold and dry it to form at least one layer of rubber film 101 on the mold. Before rolling up a pre-curling region 2 of the rubber film 101 formed on the mold, a sensing chip 4 is mounted onto the pre-curling region 2 of the rubber film 101 by a chip mounter 300. Then roll up outwards the pre-curling region 2 by a beading machine to form a cuff 3 and thus complete the manufacturing of the sheath product 100 with sensing function. Furthermore, after the chip mounter 300 mounting the sensing chip 4 onto the pre-curling region 2 of the rubber film 101 but prior to rolling up the pre-curling region 2 of the rubber film 101 on the mold, there is another mold dipping process to dip the rubber film 101 mounted with the sensing chip 4 to form at least one additional layer of rubber film. This second mold dipping process prevents the exposure of the sensing chip 4, protects the sensing chip 4 and thus improves the quality of the sheath product.

In a preferred embodiment, the sensing chip 4 is wrapped along its longitudinal direction around an exterior circumferential surface of the pre-curling region 2 of a sheath body 1. And then roll up the pre-curling region 2 with the sensing chip 4 outwards to form the cuff 3, such that the manufacture of the sheath product 100 with sensing function is completed. Wherein the sheath product 100 is made of rubber, latex or silicone and the sensing chip 4 is a flexible sensing chip.

Corresponding to the above-mentioned sheath product 100 and its manufacturing process, the present invention further provides a manufacturing equipment to embed a sensing chip in a sheath product, which is shown in FIG. 7.

The manufacturing equipment comprises a sheath-product production line. At a tail of the sheath-product production line, there is provided with, in order, a chip mounter 300 used for mounting a sensing chip 4 onto a pre-curling region 2 of a rubber film 101, and a beading machine 400 used for rolling up the pre-curling region 2 outwards to form a cuff 3. The sensing chip 4 is a flexible sensing chip, which is rolled up outwards with the pre-curling region 2.

In conclusion, the beneficial effects of the embodiments of the present invention compared to prior arts are as follows.

Firstly, the present invention adopts a process and an equipment for embedding a sensing chip, which are working with the sheath-product production line to realize automatic continuous production of sheath products. This breaks through the limitation of implementing intelligent sheath products and solves the technical issue as to embedding sensing chips into sheath products.

Secondly, the present invention adopts a protective and concealed technique to embed sensing chips into sheath products, which is working with the curling technique and the vulcanization technique to avoid any adverse influence on the appearance of the sheath products after rolling up the sensing chip into the pre-curling region. This technique is reliable as it avoids the sheath product falling off or getting damaged due to friction or impact caused during any movements occurred in usage.

Thirdly, the present invention makes it possible to collect user's physical characteristic data and/or user's movement data via the sensing chip mounted in the cuff of the sheath body. Wherein the user's physical characteristic data includes but is not limited to body temperature, pulse rate, respiration and blood pressure and the user's movement data includes but is not limited to velocity, acceleration and pressure. Such collected data are then transmitted to the intelligent terminal via wireless communication channel. Wherein the wireless communication channel comprises Bluetooth, Wi-Fi (Wireless Fidelity) and various wireless sensing techniques such as RFID (Radio Frequency Identification) etc. Users are able to obtain their physical characteristic data and/or movement data through external smart devices directly, accurately and in real time. These data enable the users to manage and adjust their body condition to control the pace of their sex life in real time, which consequently improves their experience.

The above-mentioned embodiments are the preferred embodiments of the present invention and are considered in all respects as illustrative and not restrictive. Variations and modifications are allowed within the scope of the invention. Those skilled in the art will appreciate that the features described above can be combined in various ways to form multiple variations of the invention. As a result, such variations fall within the scope of the protection to the present invention.

What is claimed is:

1. A manufacturing process of embedding a sensing chip in a sheath product comprising:
in a production line of manufacturing a sheath product, dipping a mold designed according to a shape of the sheath product in a solution of vulcanized rubber;
pulling out the mold from the solution of vulcanized rubber and drying it to form at least one layer of rubber film on the mold;
mounting a sensing chip onto a pre-curling region of the rubber film by a chip mounter before rolling up the pre-curling region of the rubber film formed on the mold; and
rolling up outwards the pre-curling region by a beading machine to form a cuff so as to complete the manufacturing of the sheath product with sensing function.

2. The manufacturing process of claim 1, characterized in that
after the chip mounter mounting the sensing chip onto the pre-curling region of the rubber film but prior to rolling up the pre-curling region of the rubber film on the mold, there is another mold dipping process in which an exterior surface of the rubber film mounted with the sensing chip is formed with at least one additional layer of rubber film.

3. The manufacturing process of claim 1, characterized in that the sensing chip is a flexible sensing chip and is rolled up outwards with the pre-curling region.

4. The manufacturing process of claim 3, characterized in that
the sensing chip is attached on the pre-curling region of the rubber film horizontally, with a longitudinal direction of the sensing chip being consistent with a circumferential direction of the pre-curling region; or
the sensing chip is attached on the pre-curling region of the rubber film vertically, with the longitudinal direction of the sensing chip being consistent with a longitudinal direction of a sheath body; or
the sensing chip is attached on the pre-curling region of the rubber film slantwise, with the longitudinal direction of the sensing chip being tilted against the longitudinal direction of the sheath body; or
the sensing chip is ring-shaped, which surrounds and attaches on an exterior circumferential surface of the pre-curling region of the rubber film.

5. A manufacturing equipment for embedding a sensing chip into a sheath product, comprising
a sheath-product production line,
at a tail of the sheath-product production line, a chip mounter configured for mounting a sensing chip onto a pre-curling region of a rubber film, and
at a tail of the chip mounter, a beading machine configured for rolling up the pre-curling region outwards to form a cuff.

6. The manufacturing equipment of claim 5, characterized in that the sensing chip is a flexible sensing chip and is rolled up outwards with the pre-curling region.

7. A sheath product with sensing function, characterized in that the sheath product comprises a sheath body, the sheath body is provided with a pre-curling region, the pre-curling region is rolled up outwards to form a cuff in which a sensing chip is embedded.

8. The sheath product with sensing function of claim 7, characterized in that the sheath body is made of rubber, latex or silicone.

9. The sheath product with sensing function of claim 8, characterized in that the sensing chip is a flexible sensing chip and is rolled up outwards with the pre-curling region, the sensing chip is an electrical element which senses user's physical characteristic data and/or user's movement data in real time and transmits such physical characteristic data and/or movement data in real time.

10. The sheath product with sensing function of claim 9, characterized in that the sensing chip comprises a power supply module, a sensing module and a data transmission module;
wherein the power supply module provides power to the sensing module and the data transmission module by self-contained power supply or wireless power supply;
the sensing module senses user's physical characteristic data and/or user's movement data; the user's physical characteristic data comprises body temperature, pulse rate, respiration, and blood pressure, while the user's movement data comprises velocity, acceleration, and pressure;
the data transmission module transmits data received from the sensing module to an intelligent terminal by wireless communication channel.

11. A data collection system, characterized in that it comprises an intelligent terminal and the sheath product with sensing function according to claim 7;
the sheath product with sensing function senses user's physical characteristic data and/or user's movement data and transmits such physical characteristic data and/or movement data to the intelligent terminal in real time;
the intelligent terminal receives user's physical characteristic data and/or user's movement data delivered by the sheath product and processes as well as displays such physical characteristic data/or movement data.

12. The data collection system of claim 11, characterized in that the intelligent terminal supplies wireless power to or charges the power supply module of the sensing chip of the sheath product.

13. A data collection system, characterized in that it comprises an intelligent terminal and the sheath product with sensing function according to claim 8;
the sheath product with sensing function senses user's physical characteristic data and/or user's movement data and transmits such physical characteristic data and/or movement data to the intelligent terminal in real time;
the intelligent terminal receives user's physical characteristic data and/or user's movement data delivered by the sheath product and processes as well as displays such physical characteristic data/or movement data.

14. The data collection system of claim 13, characterized in that the intelligent terminal supplies wireless power to or charges the power supply module of the sensing chip of the sheath product.

15. A data collection system, characterized in that it comprises an intelligent terminal and the sheath product with sensing function according to claim 9;
the sheath product with sensing function senses user's physical characteristic data and/or user's movement data and transmits such physical characteristic data and/or movement data to the intelligent terminal in real time;
the intelligent terminal receives user's physical characteristic data and/or user's movement data delivered by the sheath product and processes as well as displays such physical characteristic data/or movement data.

16. The data collection system of claim 15, characterized in that the intelligent terminal supplies wireless power to or charges the power supply module of the sensing chip of the sheath product.

17. A data collection system, characterized in that it comprises an intelligent terminal and the sheath product with sensing function according to claim 10;
the sheath product with sensing function senses user's physical characteristic data and/or user's movement data and transmits such physical characteristic data and/or movement data to the intelligent terminal in real time;
the intelligent terminal receives user's physical characteristic data and/or user's movement data delivered by the sheath product and processes as well as displays such physical characteristic data/or movement data.

18. The data collection system of claim 17, characterized in that the intelligent terminal supplies wireless power to or charges the power supply module of the sensing chip of the sheath product.
